SEQUENCE LISTING (1 sequence)

SEQ ID NO: 1 - protein, 10 aa, Artificial
  polypeptide for antigen
SLIGLGYTQT

(19) United States
(12) Patent Application Publication
(10) Pub. No.: US 2013/0280260 A1
(43) Pub. Date: Oct. 24, 2013

(54) OSTEOCLASTOGENESIS INHIBITOR CONTAINING ANTI-VDAC ANTIBODY

(71) Applicants: Shigeru Kotaka, Shinjuku-ku (JP); Yuki Nanke, Shinjuku-ku (JP); Toru Yago, Shinjuku-ku (JP); Manabu Kawamoto, Shinjuku-ku (JP); Hisashi Yamanaka, Shinjuku-ku (JP)

(72) Inventors: Shigeru Kotaka, Shinjuku-ku (JP); Yuki Nanke, Shinjuku-ku (JP); Toru Yago, Shinjuku-ku (JP); Manabu Kawamoto, Shinjuku-ku (JP); Hisashi Yamanaka, Shinjuku-ku (JP)

(73) Assignee: TOKYO WOMEN'S MEDICAL UNIVERSITY, Shinjuku-ku, Tokyo (JP)

(21) Appl. No.: 13/824,554

(22) PCT Filed: Oct. 13, 2011
(86) PCT No.: PCT/JP11/73501

§ 371 Date: Mar. 18, 2013

(30) Foreign Application Priority Data
Oct. 13, 2010 (JP) 2010-230398

(51) Int. Cl. C07K 16/28

(52) U.S. Cl. CPC C07K 16/28
USPC 424/139.1; 530/389.1; 530/387.9; 424/172.1

(57) ABSTRACT

The present invention provides a therapeutic agent or a preventative agent containing anti-VDAC antibody for diseases in which there is increased activation of osteoclasts, such as rheumatoid arthritis, and an inhibitor of human osteoclast formation containing anti-VDAC antibody.

### TECHNICAL FIELD

The present invention relates to an osteoclastogenesis inhibitor containing anti-VDAC antibody and a therapeutic agent for a disease having an increased activation of osteoclasts containing anti-VDAC antibody.

### BACKGROUND ART

A voltage-dependent anion channel (VDAC) is a pore protein, also called porin, having a molecular weight of 30 to 35 kDa. VDACs exist on the outer mitochondrial membrane, and allow molecules of 10 kDa or less to pass therethrough by free diffusion. It is known that substances are transported between mitochondria and cytoplasm (for example, Non Patent Literature 1). VDAC, together with ANT (adenine nucleotide translocase) and cyclophilin D, forms a complex called PTP (permeability transition pore). Since VDAC is involved in cell apoptosis, great attention has been focused on the function of VDAC recently (Non Patent Literature 2). Although there are a few reports that VDACs are expressed also in the cell membrane of neurons (Non Patent Literature 3), the details are still unknown. Meanwhile, the expression in the cell membrane of human osteoclasts has not been reported so far.

Osteoclasts are large multinucleated cells involved in breaking down and resorption of bones. When the bone resorption by osteoclasts exceeds bone formation by osteoblasts, abnormal bone metabolisms occur, such as osteoporosis, rheumatoid arthritis, hypercalcemia, or osteoarthritis. A disruption of the balance between osteoclasts and osteoblasts results in Paget's disease, a disorder accompanying bone breakdown.

The present inventors have found out so far that a gastric mucosal protective drug teprenone (Selbex (registered trademark)) has an inhibitory activity on human osteoclastogenesis, and have reported that the gastric mucosal protective drug is usable as a therapeutic agent or a prophylactic agent for immobilization osteoporosis (Patent Literature 1). The present inventors also have found out a 29-mer peptide derived from human rheumatoid arthritis has an inhibitory activity on human osteoclastogenesis, and have reported that the 29-mer peptide is usable as a therapeutic agent for osteoporosis or rheumatoid arthritis (Non Patent Literature 4, Patent Literature 2).

### CITATION LIST

### Patent Literatures

Patent Literature 1: Japanese Patent Application Publication No. 2005-060303
Patent Literature 2: Japanese Patent Application Publication No. 2008-148566

### Non Patent Literatures

Non Patent Literature 1: Shigeomi Shimizu, et al., Cell Technology, Vol. 18, No. 12, 1765-1772, 1999
Non Patent Literature 2: S. Shimizu, et al., The Journal of Cell, Vol. 152, 237-250, 2001.
Non Patent Literature 3: Akanda N, et al., Cell Cycle. 2008 October; 7(20): 3225-34.
Non Patent Literature 4: Kotake S, et al., Bone. 2009; 45: 627-639.

### SUMMARY OF INVENTION

### Technical Problems

An object of the present invention is to provide a novel osteoclastogenesis inhibitor.

Another object of the present invention is to provide a novel pharmaceutical composition for treating or preventing a disease having an increased activation of osteoclasts.

### Solution to Problems

As a result of earnest studies by the present inventors, the present inventors have found that VDACs are expressed in the cell membrane of human osteoclasts. The inventors have confirmed that VDACs are localized in human osteoclast membranes by immunoelectron microscopy and normal immunostaining.

Various studies have been conducted to elucidate the function of VDAC in human osteoclasts. As a result, it was found out that addition of an antibody against VDAC (anti-VDAC antibody) during the differentiation into osteoclasts from human monocytes stimulated by receptor activator NF-kappa B ligand (RANKL) not only significantly inhibits the differentiation into osteoclasts, but also inhibits the bone resorption activity of osteoclasts. Further, the patch clamp technique revealed that the anti-VDAC antibody tends to suppress a chloride current in mature human osteoclast membranes.

These results have revealed that an anti-VDAC antibody inhibits osteoclast differentiation or activation. The present invention has been made based on such findings.

Specifically, the present invention provides an osteoclastogenesis inhibitor, comprising an anti-VDAC antibody, or a Fab fragment or a F(ab′)₂ fragment thereof.

The present invention also provides a method for inhibiting osteoclastogenesis, comprising administrating an anti-VDAC antibody to a patient in need thereof.

The present invention also provides a pharmaceutical composition for treating or preventing a disease having an increased activation of osteoclasts, comprising an anti-VDAC antibody, or a Fab fragment or a F(ab′)₂ fragment thereof.

The present invention also provides a method for treating or preventing a disease having an increased activation of osteoclasts, comprising administrating an anti-VDAC antibody to a patient in need thereof.

### Advantageous Effects of Invention

The present invention can provide an osteoclastogenesis inhibitor and a pharmaceutical composition for treating a disease having an increased activation of osteoclasts, which are capable of inhibiting osteoclastogenesis and bone resorption.

### BRIEF DESCRIPTION OF DRAWINGS

FIG. 1 is an SDS-polyacrylamide gel electrophoretogram. In FIG. 1, M represents a molecular weight marker; U, unpurified; 2C8, a protein purified with a column prepared using a monoclonal antibody derived from a 5D1/3F3/2C8 line; 2H10, a protein purified with a column prepared using a monoclonal antibody derived from a 5D1/2B9/2H10 line.

FIG. 2 is a mass spectrum of Band 1.

FIG. 3 is an MS/MS spectrum of Band 1 at m/z 1456.7.

FIG. 4 is an MS/MS spectrum of Band 1 at m/z 1693.8.

FIG. 5 is an MS/MS spectrum of Band 1 at m/z 2474.2.

FIG. 6 is a graph for illustrating a probability of Band 1 based on the Mowse score (gi|238427, Mass: 30833, Score: 174, Expect: 5.5e-13). In FIG. 6, the bar at 174 includes 174, 171, 171, any of which scored VDAC hits. The hatched region is a region of random hits, and is not a significant result.

FIG. 7 is an immunoelectron micrograph showing expressions of VDACs in mitochondria and cell membrane of a human osteoclast. The lower frame in FIG. 7 shows the mitochondria, and the upper frame in FIG. 7 shows the cell membrane.

FIG. 8 is an immunoelectron micrograph showing the expression of VDACs in the mitochondria of the human osteoclast, and is an enlarged view within the lower frame in FIG. 7. In FIG. 8, dots are VDACs in the mitochondria.

FIG. 9 is an immunoelectron micrograph showing the expression of VDACs in the cell membrane of the human osteoclast, and is an enlarged view within the upper frame in FIG. 7. In FIG. 9, dots are VDACs in the cell membrane.

FIG. 10 shows nuclear staining with DAPI and immunostaining of osteoclasts with an anti-CD51/61 antibody and the anti-VDAC antibody.

FIG. 11 shows photographs of osteoclasts in control, in the presence of mouse IgG, and in the presence of the anti-VDAC antibody.

FIG. 12 shows a graph (FIG. 12-1) for illustrating a total area of osteoclasts per well in the presence of the anti-VDAC antibody or in the presence of mouse IgG; a graph (FIG. 12-2) for illustrating a total area of osteoclasts when an anti-VDAC antibody derived from a clone different from that used in FIG. 12-1; and a graph for illustrating a total area of osteoclasts in the presence or absence of a commercially-available bone resorption inhibitor (FIG. 12-3). FIG. 12-4 shows graphs created based on the data shown in FIG. 12-2, which have been statistically processed. FIG. 12-5 is the result of simultaneously examining the anti-VDAC antibody used in FIG. 12-1 and the anti-VDAC antibody used in FIG. 12-2.

FIG. 13 is a graph for illustrating the number of bone resorption pits per well in the presence of mouse IgG or in the presence of the anti-VDAC antibody.

FIG. 14 is a graph for illustrating a total area of bone resorption pits per well in the presence of mouse IgG or in the presence of the anti-VDAC antibody.

FIG. 15 shows graphs for illustrating the influence of the anti-VDAC antibody and a nonspecific antibody on a potential-dependent chloride current in human peripheral blood monocytes, mononucleated osteoclast precursor cells, and mature osteoclasts.

### DESCRIPTION OF EMBODIMENTS

An anti-VDAC antibody used in the present invention is a monoclonal antibody capable of binding to an epitope of VDAC. An antibody, which recognizes the same epitope as the anti-VDAC antibody does, can also be used in the present invention.

As the antibody used in the present invention, any antibody is applicable regardless of the class/subclass, as long as the antibody is the monoclonal antibody capable of binding to the epitope of VDAC or recognizes the same epitope as the anti-VDAC antibody does. Antibody-associated proteins linked to a Fc domain of these are also similarly applicable. Moreover, such antibodies are applicable regardless of the purity of purification raw materials. Examples thereof include naturally-occurring human antibodies, humanized antibodies and human antibodies prepared by genetic engineering, monoclonal antibodies from mice or other species, and the like.

Further, in the present invention, fragments such as a Fab fragment or a F(ab′)₂ fragment of the anti-VDAC antibody having an inhibitory activity on osteoclast differentiation or osteoclastogenesis and an inhibitory activity on osteoclast activation can also be used as in the case of the anti-VDAC antibody.

The anti-VDAC antibody may be produced from osteoclast membrane proteins by known methods, or commercially-available products may also be used. The commercially-available products are sold from, for example, Invitrogen Corporation, Calbiochem, and Santa Cruz Biotechnology, Inc.

An epitope for the antibody used in the present invention is preferably composed of 20 or less amino acid residues. The epitope is more preferably composed of 10 or less amino acid residues. The epitope further preferably contains an amino acid sequence of SEQ ID NO: 1 as follows.

(SEQ ID NO: 1)
Ser Leu Ile Gly Leu Gly Tyr Thr Gln Thr
1 5 10

The anti-VDAC antibody is administered directly or in the form of various pharmaceutical compositions.

The administration target is animals including human, for which a treatment for a disease having an increased activation of osteoclasts is required, or osteoclastogenesis inhibition is required. Examples of the disease having an increased activation of osteoclasts include osteoporosis, rheumatoid arthritis, Paget's disease, and osteopenic and opsteoclastic diseases such as bone metastases of cancers (lung cancer, breast cancer, prostate cancer, or the like).

The osteoclasts are preferably human osteoclasts.

The dosage form of the pharmaceutical composition is desirably for subcutaneous injection or intravenous drip injection. These dosage forms can be produced according to conventional methods.

The anti-VDAC antibody is administered normally once or multiple times a day to an adult at a dosage of 1000 to 10000 μg/kg body weight, preferably 3000 to 5000 μg/kg body weight, based on purified protein.

The dose of a therapeutic agent or an inhibitor of the present invention to be administered is determined, depending on the administration route, treatment period, the age and body weight of a patient, and so forth.

### EXAMPLE 1

### 1. Identification of Novel Human Osteoclast Membrane Surface Proteins

(1) Preparation of Monoclonal Antibodies against Cell Membrane Proteins of Osteoclasts
(i) Peripheral blood monocytes from five healthy subjects were cultured in flasks in the presence of M-CSF and RANKL for approximately 2 weeks and differentiated into osteoclasts. After trypsin and EDTA were added to the human osteoclasts, the resulting cells were cultured at 37° C. for 15 minutes, and then detached with a cell scraper and harvested. This operation was repeated to collect cell membranes of human mature osteoclasts. Membrane proteins were prepared from the human mature osteoclasts using a RIPA buffer.
(ii) Using the obtained membrane proteins as an antigen, mice were immunized. Spleen cells of the mice thus immunized were fused with myeloma cells, and four types of hybridomas were obtained. Screening was performed by ELISA using a plate on which the osteoclast membrane proteins were immobilized. The wells of the positive result were further examined for a reactivity to human osteoclasts and a nonspecific reactivity to monocytes. After cloning, 5D1/2B9/2H10 and 5D1/3F3/2C8 were obtained as final lines.
(iii) From these final lines, monoclonal antibodies were obtained.
(2) Purification of Proteins Bound to Antibody Column

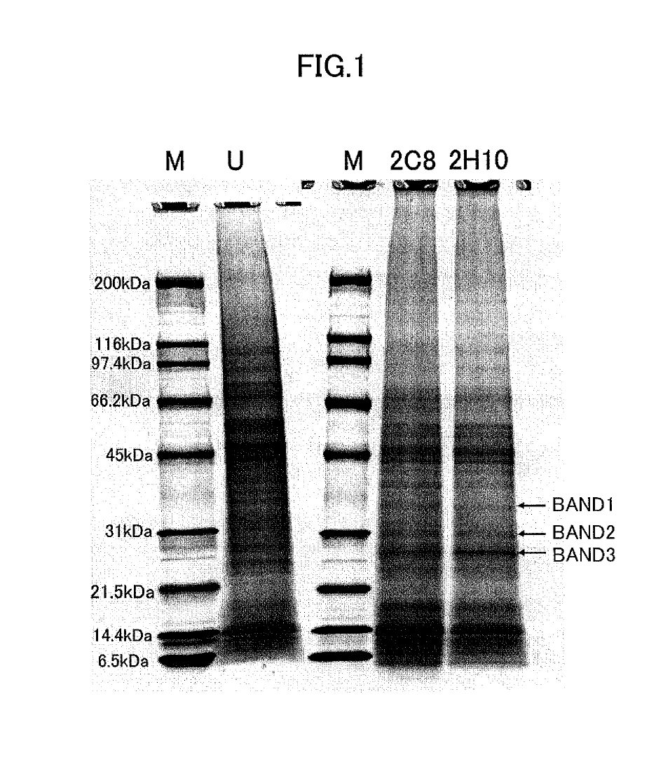

The monoclonal antibody (invitrogen A31855 anti-porin, human mitochondrial, mouse IgG2b, monoclonal 31HL) obtained above was fixed to a column, and an antibody column was prepared. A purification operation was performed on fractions bound to the antibody column from the osteoclast membrane proteins.
(3) Identification of Purified Proteins
(i) SDS-polyacrylamide gel electrophoresis (CBB staining) and western blotting were performed on the obtained fractions. As a result of the two, 3 types of proteins were obtained, which were expected to be purified with the antibody column (FIG. 1). Of the 3 types of the proteins, one with 36 kDa was designated by Band 1; 31 kDa, Band 2; 28 kDa, Band 3.
(ii) These proteins were cleaved with a digestive enzyme (Achromobacter protease I (lysyl endopeptidase, Lys-C, Wako Pure Chemical Industries, Ltd.)), and subjected to in-gel digestion. Then, mass spectra of the digested products were obtained. Further, MS/MS spectra of Band 1 were obtained at m/z 1456.7, m/z 1693.8, and m/z 2474.2. Subsequently, a peak list was created, and the measured data was compared with predicted data calculated from the registered sequence using software (MASCOT, manufactured by Matrix Science Limited). The identification probability was evaluated according to the probability-based Mowse score. As a result of the peptide mass fingerprinting (PMF), one of the proteins was finally identified as VDAC. The same experiment was conducted on Bands 2 and 3, but it was difficult to identify these proteins.

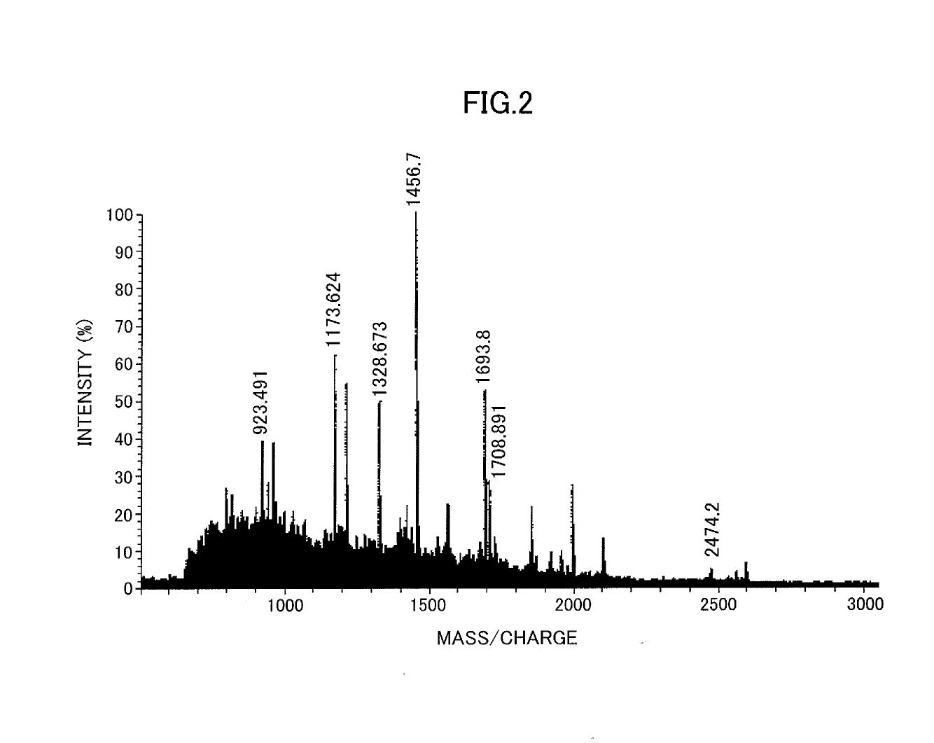

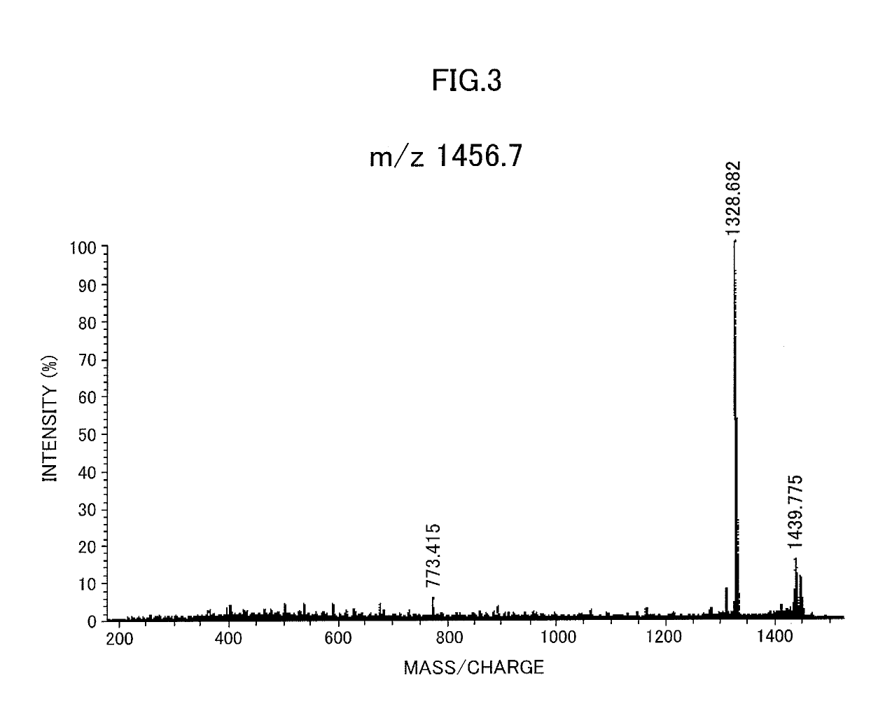

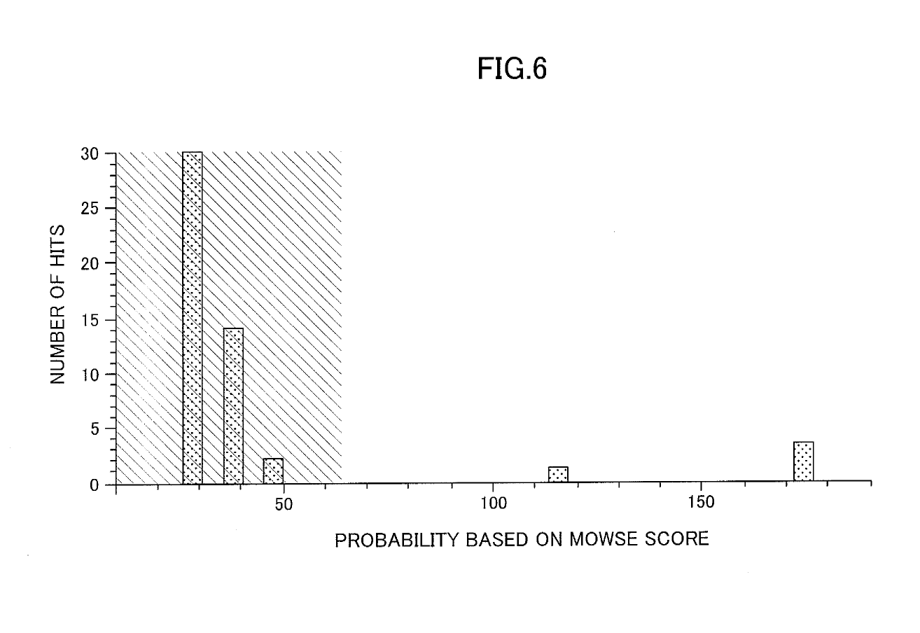

FIG. 2 shows the mass spectrum of Band 1 (Bands 2, 3 are not illustrated). FIGS. 3 to 5 show the MS/MS spectra of Band 1 at m/z 1456.7, m/z 1693.8, and m/z 2474.2. FIG. 6 shows the probability of Band 1 based on the Mowse score.

### 2. Expressions of VDAC in Mitochondria and Cell Membrane of Osteoclast

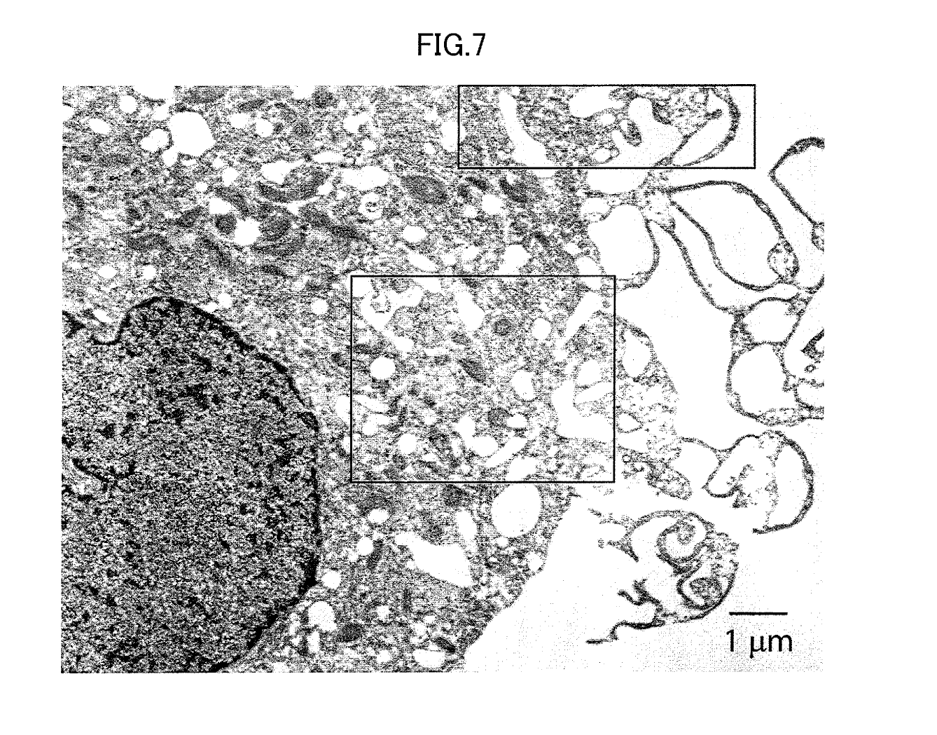

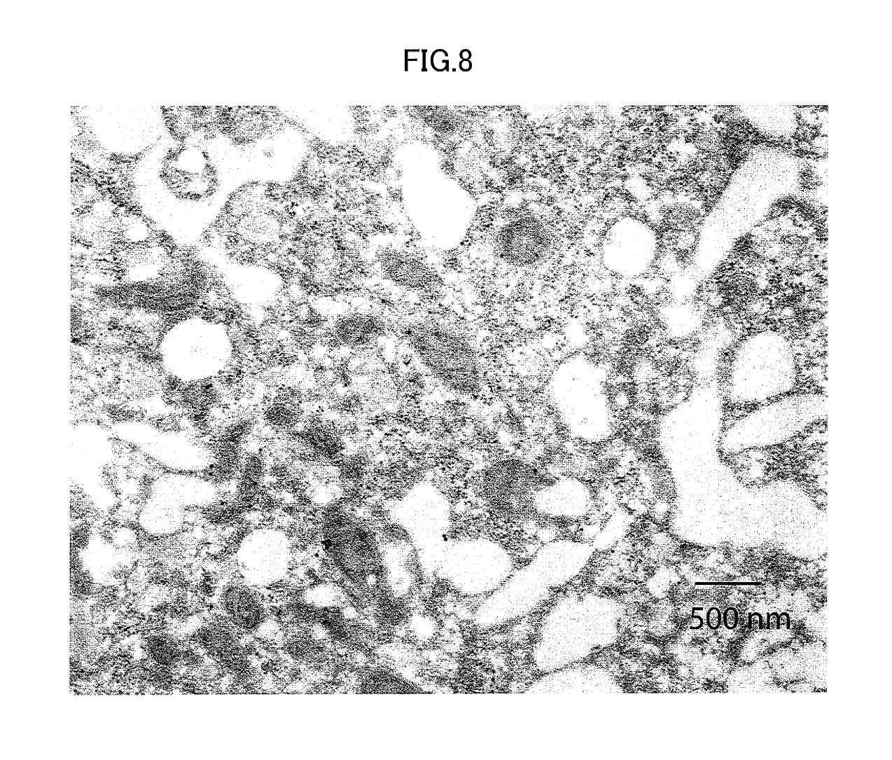

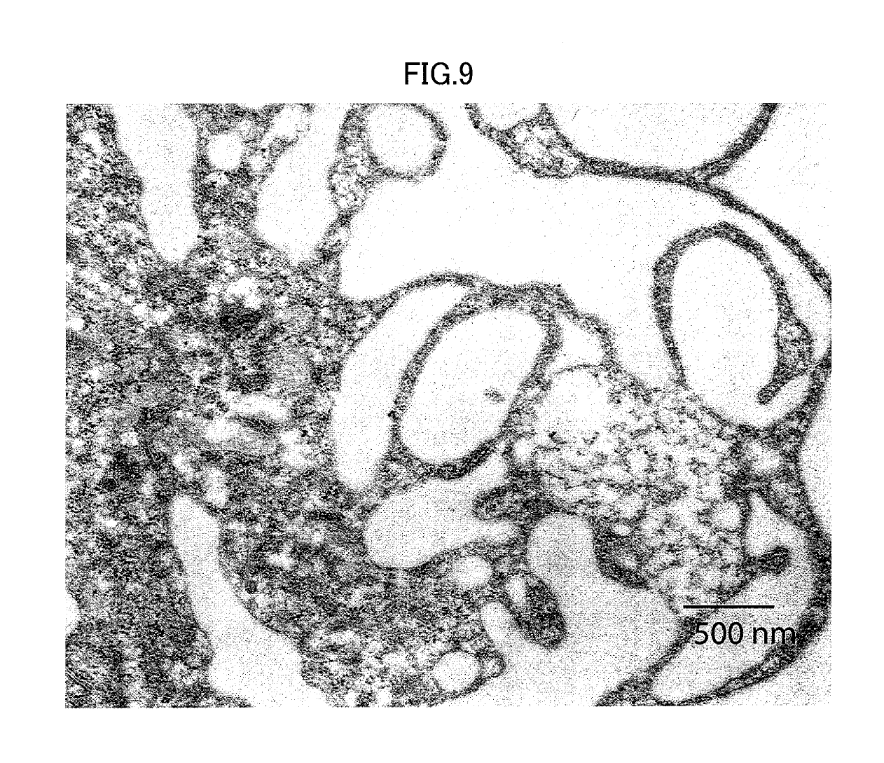

The expression of VDACs in a human osteoclast was examined with an immunoelectron microscope. As a result, the expressions of VDACs were observed in mitochondria (FIG. 7; lower frame, FIG. 8; dots) and in the cell membrane (FIG. 7; upper frame, FIG. 9; dots).

### 3. Confirmation of Localization of VDAC in Osteoclast Membrane

Nuclear staining with DAPI and immunostaining of osteoclasts with an anti-CD51/61 antibody and the anti-VDAC antibody confirmed the localization of VDACs in the osteoclast membrane.

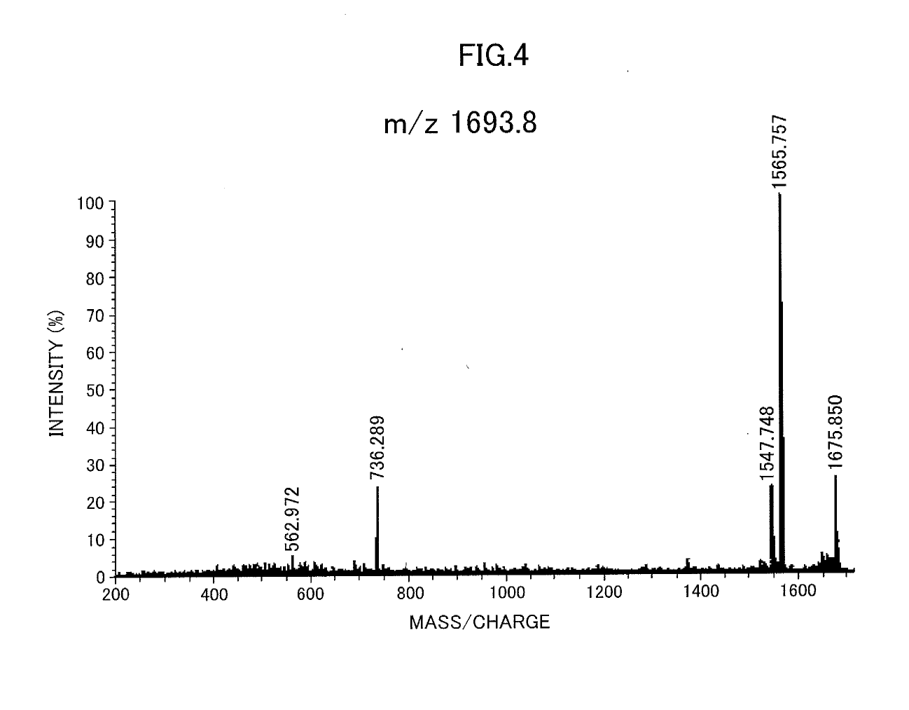

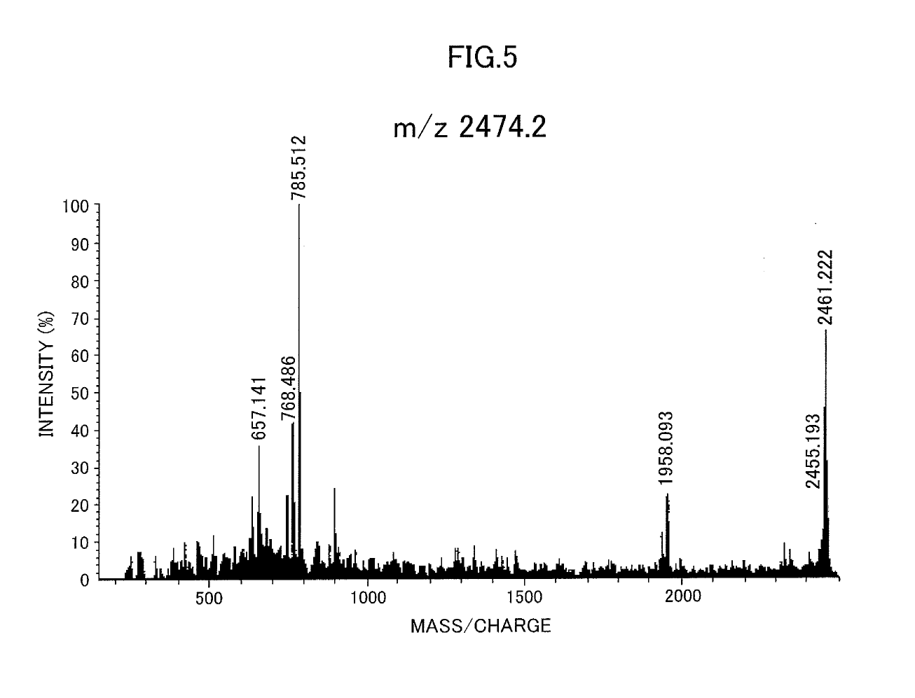

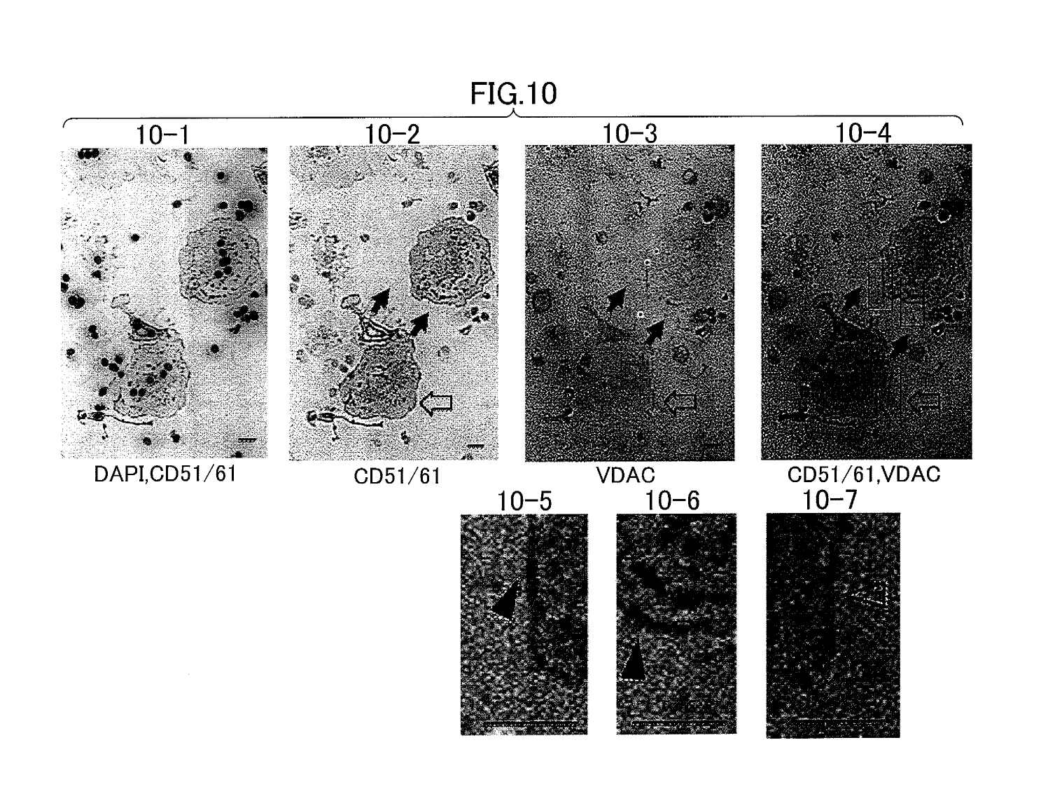

In the multinucleated osteoclasts (FIG. 10-1; the nuclei were stained blue with DAPI, bar=10 μm), the superimposition of CD51/61 (FIG. 10-2; green) and VDAC (FIG. 10-3; red) exhibited yellow portions as a result of the superimposition of red and green (FIG. 10-4; three frames, FIGS. 10-5, 10-6, and 10-7; enlarged images of the frames, pointed by the arrows). This suggested the presence of a membrane portion where CD51/61 and VDAC were co-expressed.

### 4. Inhibition of Osteoclast Differentiation and Osteoclastogenesis by Anti-VDAC Antibody

(1) Into the human osteoclastogenesis culture system obtained in step 1. (1) (i) above, the anti-VDAC antibody (5 μg/ml, invitrogen A31855 anti-porin, human mitochondrial, mouse IgG2b, monoclonal 31HL) was added. Nothing was added in a control. As a comparison substance, mouse IgG was added in the same amount (n=4).

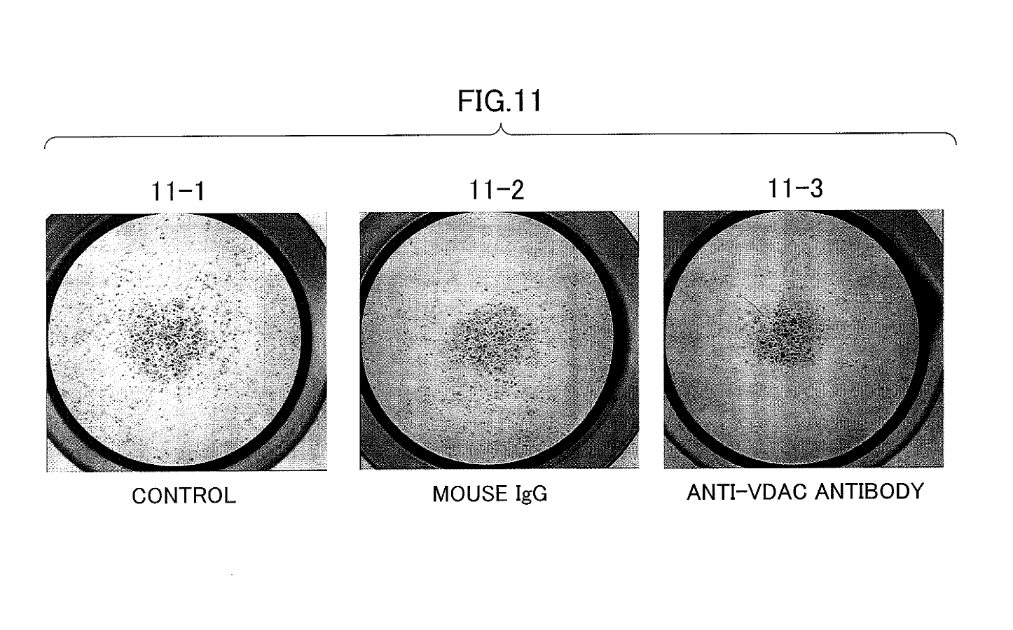

As a result, the osteoclastogenesis was significantly inhibited when the anti-VDAC antibody was added (FIGS. 11-3, 12-1) in comparison with the control (FIG. 11-1) and mouse IgG (FIGS. 11-2, 12-1).
(2) The inhibition of osteoclast differentiation and osteoclastogenesis was evaluated in the same manner as in (1) above, except that an anti-VDAC antibody (VDAC1 (20B12), SANTA CRUZ Biotechnology, Inc., sc-58649, mouse monoclonal IgG2b, 100 μg/ml) derived from a different clone was used, and that peripheral blood was obtained from two healthy subjects (n=4).

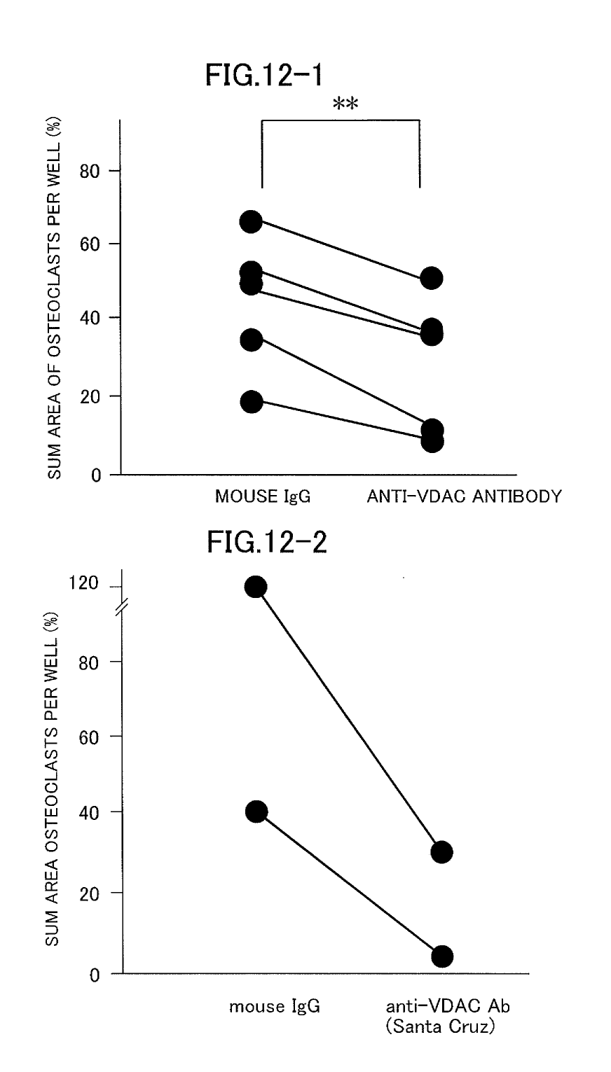

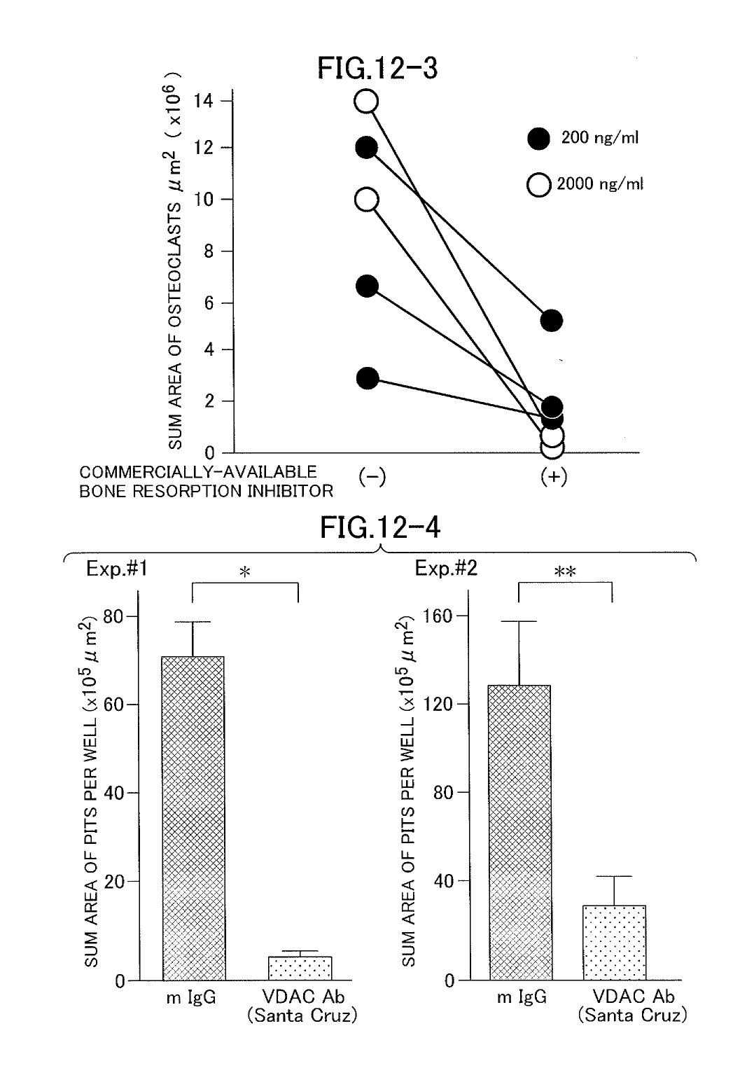

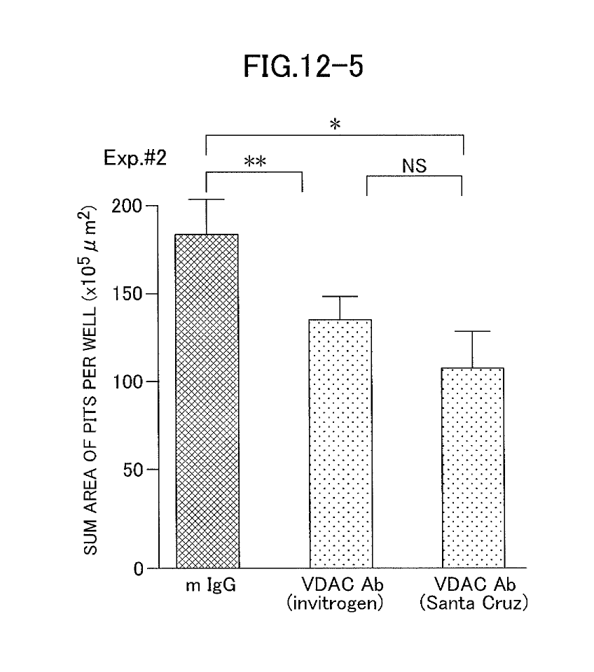

As a result, when the anti-VDAC antibody derived from the different clone was added, the osteoclastogenesis was also significantly inhibited (FIG. 12-2) in comparison with the control.
(3) The inhibition of osteoclast differentiation and osteoclastogenesis was evaluated in the same manner as in (1) above, except that a commercially-available bone resorption inhibitor (Teij in Pharma Limited, brand name Teiroc (registered trademark) injection 5 mg, active ingredient: generic name alendronate sodium hydrate) was added in an amount of 0.2 μg/mL or 2 μg/mL in place of the anti-VDAC antibody (5 μg/ml). Nothing was added in a control.

As a result, when the commercially-available bone resorption inhibitor was added, the osteoclastogenesis was significantly inhibited (FIG. 12-3) in comparison with the control.

When FIGS. 12-1, 12-2 are compared with FIG. 12-3, the slopes of lines in the graphs are substantially the same. Accordingly, it can be stated that the anti-VDAC antibodies demonstrated substantially the same or higher inhibitory effect on osteoclast differentiation and osteoclastogenesis than the existing bone resorption inhibitor.
(4) Graphs created based on the data obtained in (2) above and statistically processed are presented (FIG. 12-4). *p<0.0001, **p=0.0009.
(5) Further, the inhibition of osteoclast differentiation and osteoclastogenesis was examined in the same manner as in (1) above, except that the anti-VDAC antibody used in (1) and the anti-VDAC antibody used in (2) were simultaneously used (FIG. 12-5). *p=0.0147, **p=0.0421.

### 5. Inhibition of Bone Resorption Activity of Osteoclasts

Using a bone culture system (manufactured by BD Biosciences, BD BioCoat™ Osteologic™), the bone resorption ability of osteoclasts was evaluated based on resorption pits. A culture vessel of the system is coated with a synthetic calcium phosphate thin film as a bone alternative. When osteoclasts are cultured on this coat, bone resorption pits are formed on the thin film, and the bone resorption ability can be evaluated.

Into the human osteoclastogenesis culture system obtained in step 1. (1) (i) above, the anti-VDAC antibody (5 μg/ml) was added. As a positive control, a mouse IgG antibody was added in the same amount. After each of the culture systems was subjected to culturing at 37° C. for 14 days, the number and total area of bone resorption pits were determined using a fluorescence microscope BZ-9000 (Keyence Corporation).

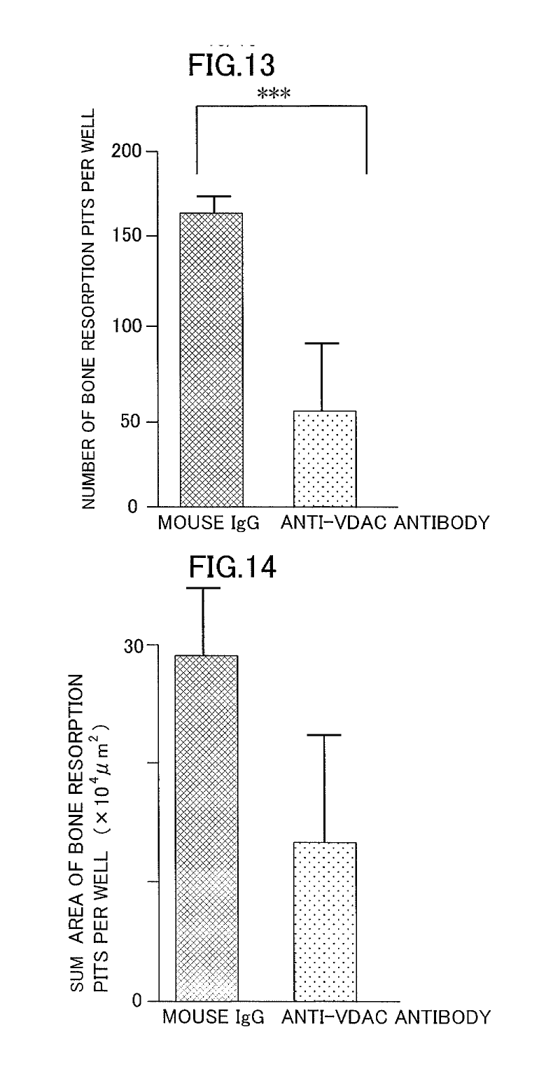

As a result, as in the case of the osteoclast differentiation and osteoclastogenesis, when the anti-VDAC antibody was added, significant inhibitions of the number and total area of bone resorption pits were observed in comparison with mouse IgG, (FIGS. 13, 14).

### 6. Influence of Anti-VDAC Antibody on Potential-Dependent Chloride Current in Mature Osteoclasts

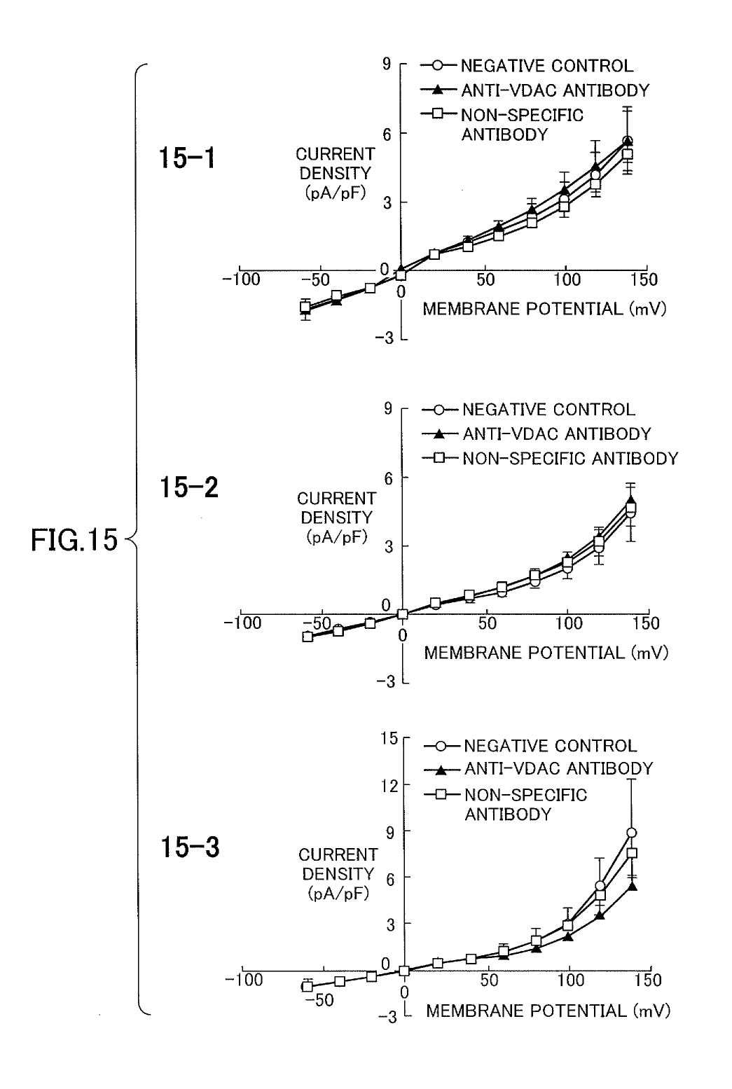

The influence of the anti-VDAC antibody (5 μg/ml) (FIG. 15, ▴) on a potential-dependent chloride current in human peripheral blood monocytes, mononucleated osteoclast precursor cells, and mature osteoclasts was examined using the patch clamp technique. The same method was performed on an extracellular fluid (FIG. 15, ∘) as a negative control and on a nonspecific antibody Non-specific rabbit IgG (R&D 20116, mouse IgG2b isotype control) (FIG. 15, □) as a comparison substance.

The anti-VDAC antibody did not influence a corrected value for the potential-dependent chloride current strength in the human peripheral blood monocytes (FIG. 15-1) and the mononucleated osteoclast precursor cells (FIG. 15-2). Meanwhile, the anti-VDAC antibody showed to have a tendency to decrease the potential-dependent chloride current in the mature osteoclasts (FIG. 15-3) in comparison with the nonspecific antibody.

The above result demonstrated that the anti-VDAC antibody has a tendency to actually decrease the potential-dependent chloride current in mature osteoclasts.

### 7. Analysis of Epitope for Anti-VDAC Antibody

In order to examine an epitope for the anti-VDCA monoclonal antibody (VDAC1(20B12), SANTA CRUZ Biotechnology, Inc., sc-58649, mouse monoclonal IgG2b, 100 μg/ml), an analysis was conducted by ELISA using synthetic peptides (Table 1). As the synthetic peptides, 28 types of peptides were prepared so as to cover the VDAC1 completely.

### 7.1. Fixation of Synthetic Peptides to Plates

The synthetic peptides were fixed to plates by an avidin-biotin method as follows.

The synthetic peptides (325 nmol/peptide) were each dissolved in 250 μL of DMSO, and then diluted 6400-fold with Wash Buffer (PBS containing 0.05% Tween-20). After a streptavidin-coated plate was washed with Wash Buffer (350 μL, 3 times), 100 μL of the peptide thus diluted was added into the plate, which was left standing in cold storage overnight.

7.2. Detection of Antibodies Reacting with Peptides by ELISA

ELISA was performed using the plate to which the peptide was fixed in 7.1, and antibodies bound to the peptide were detected. The detection procedure was as follows. Two wells were used for one condition, and the average was taken.
(1) The liquid in wells was taken out. After washing with Wash Buffer (350 μL, three times), 200 μL of Blocking Buffer (SynBlock) was added to the wells, which were shaken at 37° C. for 1 hour.
(2) The liquid in the wells was taken out, and 50 μL of the monoclonal antibody 20B12 (diluted to 5 μg/mL with Blocking Buffer) was added to the wells, which were shaken at 37° C. for 1 hour.
(3) The liquid in the wells was taken out. After washing with Wash Buffer, 50 μL of HRP-labeled Goat anti Mouse IgG (Fc) (the original solution had been diluted 25000-fold with Wash Buffer) was added to the wells, which were shaken at 37° C. for 1 hour.
(4) The liquid in the wells was taken out, and the plate was washed. Then, 60 μL of a staining solution was added to the plate, which was incubated at 37° C. for 30 minutes. After 60 μL of a stop solution was added thereto to stop the color reaction, the absorbance at 450 nm (Reference 595 nm) was measured with a plate reader.

Used as a positive control was a constituent peptide of interferon β, which reacted with an interferon β monoclonal antibody. Used as a negative control was a constituent peptide of the same interferon β, which did not react with the antibody. The detection procedure was as follows.
(1) The liquid in wells was taken out. After washing with Wash Buffer (350 μL, three times), 200 μL of Blocking Buffer was added to the wells, which were shaken at 37° C. for 1 hour.
(2) The liquid in the wells was taken out. After washing with Wash Buffer, 100 μL of an HRP-labeled anti-interferon β monoclonal antibody (dissolved in Wash Buffer,3 mL/vial) was added to the wells, which were shaken at 37° C. for 1 hour.
(3) The liquid in the wells was taken out, and the plate was washed. Then, 60 μL of a staining solution was added to the plate, which was incubated at 37° C. for 30 minutes. After 60 μL of a stop solution was added thereto to stop the color reaction, the absorbance at 450 nm (Reference 595 nm) was measured with a plate reader.

TABLE 1
Amino acid sequences of
synthesized peptides

| Peptide No. | Sequence |
|---|---|
| 1 | MAVPPTYADL GKSARDVFTK |
| 2 | GKSARDVFTK GYGFGLIKLD |
| 3 | GYGFGLIKLD LKTKSENGLE |
| 4 | LKTKSENGLE FTSSGSANTE |
| 5 | FTSSGSANTE TTKVTGSLET |
| 6 | TTKVTGSLET KYRWTEYGLT |
| 7 | KYRWTEYGLT FTEKWNTDNT |
| 8 | FTEKWNTDNT LGTEITVEDQ |
| 9 | LGTEITVEDQ LARGLKLTFD |
| 10 | LARGLKLTFD SSFSPNTGKK |
| 11 | SSFSPNTGKK NAKIKTGYKR |
| 12 | NAKIKTGYKR EHINLGCDMD |
| 13 | EHINLGCDMD FDIAGPSIRG |
| 14 | FDIAGPSIRG ALVLGYEGWL |
| 15 | ALVLGYEGWL AGYQMNFETA |
| 16 | AGYQMNFETA KSRVTQSNFA |
| 17 | KSRVTQSNFA VGYKTDEFQL |
| 18 | VGYKTDEFQL HTNVNDGTEF |
| 19 | HTNVNDGTEF GGSIYQKVNK |
| 20 | GGSIYQKVNK KLETAVNLAW |
| 21 | KLETAVNLAW TAGNSNTRFG |
| 22 | TAGNSNTRFG IAAKYQIDPD |
| 23 | IAAKYQIDPD ACFSAKVNNS |
| 24 | ACFSAKVNNS SLIGLGYTQT |
| 25 | SLIGLGYTQT LKPGIKLTLS |
| 26 | LKPGIKLTLS ALLDGKNVNA |
| 27 | ALLDGKNVNA GGHKLGLGLE |
| 28 | GGHKLGLGLE FQA |

Tables 2, 3 show the absorbance data. Reactions were observed between peptides 24, 25 and the antibodies, while the reactions of the others were almost at a blank level. Particularly, peptide 24 was significant, and it is believed that the sequence thereof contains that of the epitope. Peptides 24 and 25 overlap each other by 10 residues, and a weak reaction was also observed from peptide 25 also. Accordingly, it is believed that some residues among the 10 overlapping residues form a portion of the epitope. It has been reported that the steric structure of a VDAC protein is cylindrical (Hiller, S., et al., Science, 2008, Vol. 321: 1206-1210). From the reported steric structure and the position of the 10 residues specified this time, it was found out that the 10 overlapping residues between peptides No. 24 and No. 25 are located on the side surface of the cylindrical shape. This location seems to be a location recognizable by an antibody administered from the outside.

TABLE 2
Table 2-1 Result of ELISA using synthetic peptides (first time)

| | Peptide | | | | | | | | | |
|---|---|---|---|---|---|---|---|---|---|---|
| | 1 | 2 | 3 | 4 | 5 | 6 | 7 | 8 | 9 | 10 |
| OD₄₅₀₋₅₉₅ | 0.448 | 0.363 | 0.280 | 0.312 | 0.311 | 0.358 | 0.321 | 0.341 | 0.286 | 0.360 |
| | Peptide | | | | | | | | | |
| | 11 | 12 | 13 | 14 | 15 | 16 | 17 | 18 | 19 | 20 |
| OD₄₅₀₋₅₉₅ | 0.319 | 0.345 | 0.369 | 0.316 | 0.337 | 0.388 | 0.285 | 0.275 | 0.300 | 0.362 |

| | Peptide | | | | | | | | |
|---|---|---|---|---|---|---|---|---|---|
| | 21 | 22 | 23 | 24 | 25 | 26 | 27 | 28 | No peptide* |
| OD₄₅₀₋₅₉₅ | 0.235 | 0.318 | 0.276 | 1.284 | 0.590 | 0.268 | 0.290 | 0.418 | 0.354 |

Table 2-2 Result of ELISA using synthetic peptides (first time)

| | Negative | Positive | |
|---|---|---|---|
| Peptide | control | control | No peptide |
| OD₄₅₀₋₅₉₅ | 0.063 | 1.144 | 0.063 |

*Average of n = 8 was taken

TABLE 3
Table 3-1 Result of ELISA using synthetic peptides (second time)

| | Peptide | | | | | | | | | |
|---|---|---|---|---|---|---|---|---|---|---|
| | 1 | 2 | 3 | 4 | 5 | 6 | 7 | 8 | 9 | 10 |
| OD₄₅₀₋₅₉₅ | 0.443 | 0.421 | 0.354 | 0.423 | 0.351 | 0.363 | 0.371 | 0.411 | 0.338 | 0.351 |
| | Peptide | | | | | | | | | |
| | 11 | 12 | 13 | 14 | 15 | 16 | 17 | 18 | 19 | 20 |
| OD₄₅₀₋₅₉₅ | 0.361 | 0.375 | 0.322 | 0.385 | 0.373 | 0.444 | 0.308 | 0.305 | 0.312 | 0.398 |

| | Peptide | | | | | | | | |
|---|---|---|---|---|---|---|---|---|---|
| | 21 | 22 | 23 | 24 | 25 | 26 | 27 | 28 | No peptide* |
| OD₄₅₀₋₅₉₅ | 0.270 | 0.319 | 0.357 | 1.336 | 0.679 | 0.367 | 0.371 | 0.455 | 0.397 |

Table 3-2 Result of ELISA using synthetic peptides (second time)

| | Negative | Positive | |
|---|---|---|---|
| Peptide | control | control | No peptide |
| OD₄₅₀₋₅₉₅ | 0.119 | 1.329 | 0.109 |

*Average of n = 8 was taken

As described above, by employing orthodox immunological methods such as immunization of mice with membrane proteins and productions of hybridomas and monoclonal antibodies, it was successfully revealed for the first time that VDACs normally expressed only in mitochondria are expressed in human osteoclast membranes.

Further, the localization in the cell membranes was successfully demonstrated by the immunoelectron microscopy and the normal immunostaining method. Additionally, the antibody against this VDAC was shown to have a tendency to decrease a chloride current in mature osteoclasts. Furthermore, the anti-VDAC antibody inhibited the osteoclastogenesis from human monocytes stimulated by RANKL.

The foregoing has suggested potential applications of this antibody in the future to treatments for diseases having an increased activation of osteoclasts, such as, for example, osteoporosis, rheumatoid arthritis, and Paget's disease, or bone metastases of lung cancer, breast cancer, prostate cancer, and the like.

1-15. (canceled)

16. An osteoclastogenesis inhibitor comprising an anti-VDAC antibody, or a Fab fragment or a F(ab′)₂ fragment thereof.

17. The osteoclastogenesis inhibitor according to claim 16, wherein osteoclasts are human osteoclasts.

18. The osteoclastogenesis inhibitor according to claim 16, wherein an epitope for the anti-VDAC antibody comprises 20 or less amino acid residues.

19. The osteoclastogenesis inhibitor according to claim 16, wherein an epitope for the anti-VDAC antibody comprises an amino acid sequence of SEQ ID NO: 1.

20. A method for inhibiting osteoclastogenesis, comprising an administrating an anti-VDAC antibody to a patient in need thereof.

21. The method according to claim 20, wherein an epitope for the anti-VDAC antibody comprises 20 or less amino acid residues.

22. The method according to claim 20, wherein an epitope for the anti-VDAC antibody comprises an amino acid sequence of SEQ ID NO: 1.

23. A pharmaceutical composition for treating or preventing a disease having an increased activation of osteoclasts, comprising an anti-VDAC antibody, or a Fab fragment or a F(ab′)₂ fragment thereof.

24. The pharmaceutical composition according to claim 23, wherein the disease having an increased activation of osteoclasts is selected from the group consisting of osteoporosis, rheumatoid arthritis, Paget's disease, and bone metastases.

25. The pharmaceutical composition according to claim 23, wherein the osteoclasts are human osteoclasts.

26. The pharmaceutical composition according to claim 23, wherein an epitope for the anti-VDAC antibody comprises 20 or less amino acid residues.

27. The pharmaceutical composition according to claim 23, wherein the epitope for the anti-VDAC antibody comprises an amino acid sequence of SEQ ID NO: 1.

28. A method for treating or preventing a disease having an increased activation of osteoclasts, comprising an administrating an anti-VDAC antibody to a patient in need thereof.

29. The method according to claim 28, wherein an epitope for the anti-VDAC antibody comprises 20 or less amino acid residues.

30. The method according to claim 28, wherein the epitope for the anti-VDAC antibody comprises an amino acid sequence of SEQ ID NO: 1.
